SEQUENCE LISTING (1 sequence)

SEQ ID NO: 1 - DNA, 2043 bp, Homo sapiens
atgatcaacatgggtgatagtcatgtcgatacttcctctactgtatctgaagctgttgctgaagaagtctctctattctctatgactgacatgattctgttctcactgattgttggactgttgacatactggttcttattccgcaaaaagaaagaggaagtaccagaattcaccaagatccaaacacttaccagttctgtacgtgaaagttcctttgttgagaaaatgaagaaaacaggtcgaaacatcattgtgttctacggttctcaaacaggtactgcagaagaattcgctaatcgactttccaaagatgctcatcgttatggaatgagaggtatgagtgctgatcctgaagaatacgacttagccgatttatcgagtcttccagaaatcgacaatgctttggttgtgttctgtatggctacttatggagaaggagatcctactgataacgctcaagacttctacgattggttacaggaaactgatgtcgatttatctggcgtaaagtttgcagtgtttggactaggtaacaagacttacgagcatttcaatgctatgggtaaatacgtggacaaaaggttggagcaattaggtgcacaacgcattttcgagttaggtttaggagatgatgacggaaatctagaagaggacttcatcacttggagagaacagttttggttagcagtatgtgaacactttggagttgaagcaactggtgaagaaagctctattcgacagtatgaactagtcgttcatactgacattgatgctgcaaaggtgtatatgggagaaatgggaagactgaaatcttacgagaatcagaaaccaccttttgacgcaaagaatcccttcttagcagctgttacaaccaatcgcaaactgaaccaaggtacagaacgtcatcttatgcacttagaactggacatttcagactcgaagattcgttacgaatctggtgatcatgttgctgtttatcctgcaaatgactctgctttagtcaaccaattgggtaagattcttggtgcagacttagatgtcgtaatgagtctgaacaatttggatgaagaatctaacaagaaacacccttttccatgtccaacttcctataggactgctttgacctactatctggatatcacgaatccacctcgtacaaacgtcctttatgaactagctcaatacgcttctgaaccttctgaacaagagctattgcgaaaaatggcatcatcttctggtgaaggaaaggagttgtatctgtcttgggtagttgaagcaagacgacatatacttgccattctgcaagattgtccttcattacgaccacctatagatcacctatgcgaattacttcccagattacaagcacgttactattccatagcaagttctagcaaagttcatccaaactcagttcacatttgtgctgttgttgttgaatacgagactaaagctggtcgtatcaacaaaggtgttgctacgaattggttacgtgctaaagaacctgttggtgaaaatggtggtcgtgctttagtcccaatgtttgttcgtaagagccaattcaggttacctttcaaagcaacaactcctgtgattatggtaggtcctggtactggtgttgcacctttcattggcttcatccaagaaagagcatggttacgtcaacaaggaaaggaagttggtgaaaccctactttactatggatgtcgtcgttcagatgaagattacttgtatcgagaagagttagctcagtttcatcgtgatggtgctttaacgcaacttaacgtggctttttctcgtgaacaatcgcacaaagtttacgttcagcatttgcttaagcaagatcgtgaacacttatggaagctaatcgaaggaggtgctcacatctatgtctgtggtgatgctaggaatatggctcgtgatgtacagaacacattctacgacatagttgctgaattaggtgctatggaacatgctcaagctgttgattacatcaagaaactgatgacgaaaggaagatactctctggatgtttggagctaa

(19) United States
(12) Patent Application Publication
(10) Pub. No.: US 2011/0045550 A1
(43) Pub. Date: Feb. 24, 2011

(54) Fission Yeast Expressing Cytochrome P450 Reductase

(75) Inventor: Calin-Aurel Dragan, Saarbrucken (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: POMBIOTECH GMBH, Saarbrucken (DE)

(21) Appl. No.: 12/664,031

(22) PCT Filed: Jun. 12, 2008
(86) PCT No.: PCT/EP2008/057371

§ 371 Date: Apr. 27, 2010

(30) Foreign Application Priority Data
Jun. 12, 2007 (EP) 07011515.9

(51) Int. Cl. C12P 17/14; C12N 1/19; C12N 9/02; C12P 1/02; C12P 13/00; C12P 17/10; C12P 17/12

(52) U.S. Cl. 435/120; 435/254.2; 435/189; 435/171; 435/128; 435/121; 435/122

(57) ABSTRACT

The present invention refers to a recombinant fission yeast strain useful for the improved production of human cytochrome P450s. The improvement in the cytochrome P450 rate is achieved by a coexpression of a functional human electron transfer chain e.g. CPR (cytochrome P450 reductase). The invention further relates to uses of the recombinant fission yeast and particularly the use of the recombinant fission yeast in methods to generate cytochrome P450 dependent metabolites.

Reference metabolite standards of hit or lead pharmaceutical compounds or toxic substances are needed for their structural confirmation and pharmacological or toxicological characterization, including studies on their pharmacodynamic/-kinetic properties, on enzyme kinetics of their formation, and on phase II metabolism. However, such metabolite standards are often not commercially available, particularly in the case of new (designer) drugs. The classical chemical synthesis of drug metabolites can be cumbersome and stereo-chemically demanding and hence go beyond the possibilities of most biochemistry or pharmacology/toxicology oriented laboratories. Custom-made metabolite standards are a possible but usually very expensive solution.

Accordingly, there is still an already long existing need to provide methods for the production of clinically acceptable reference standards and drug metabolites.

One alternative is to generate such acceptable reference standards and drug metabolites by in vitro methods using purified human cytochrome P450 liver enzymes. Thus, since the beginning of research on human P450s, heterologous expression systems were invented in order to circumvent the tedious purification of human enzymes from liver tissue and availability of human livers or microsomal preparations.

Several groups used mammalian cell culture systems of either transiently transfected or stably transfected cell lines in order to provide the suitable eukaryotic environment for the heterologous expression of human liver P450s. Although studies on metabolism were now set free from the firstly mentioned constraints, cost and time requirements were relatively high while the yield of enzyme was quite low.

Soon microorganisms became a new target for the expression of human enzymes. Escherichia coli soon became one option due to very high expression levels between 50 and 1500 nmol of enzyme per litre of culture. Unfortunately, due to the membrane protein character of P450s minor to major amino acid sequence modifications of P450s were required in order to obtain high expression levels. Remarkably, bacterial systems could be made more functional by coexpression of human CPR both, in vivo and in vitro. Blake et al. (1996) reported the coexpression of human CPR in bacterial systems (see Tab. 1).

Also bakers yeast became a host system for the expression of chytochrome P450s. It has the great advantage of offering a well-developed intracellular membrane and compartment system. In this system it seemed that the bakers yeast cytochrome P450 reductase (yCPR), particularly when overexpressed, worked well with heterologous human P450s.

It is to note that most of the work carried out on heterologous expression systems (see Tab. 1) was mainly done in order to obtain the pure enzyme or even only the membrane fraction (microsomes). However, there is so far no heterologous expression system for the expression of cytochrome P450, which would be useful for or allow an up-scaling for the biosynthesis of pharmaceutically interesting metabolites.

TABLE 1
Selection of heterologous expression systems for human liver P450s.
For the hosts the following abbreviations are used:
Eco = Escherichia coli, Sce = Saccharomyces cerevisiae, Sf9 = Spodoptera frugiperda.

| Host | P450¹ | mod² | CPR³ | carried out in | Reference |
|---|---|---|---|---|---|
| Sce | 3A4 | no | yeast, coexp. | vitro | Brian et al. (1990) |
| Sce | 3A4 | no | yeast, coexp. | vitro | Peyronneau et al. (1992) |
| Sce | 2D6 | no | yeast, coexp. | vitro | Ellis et al. (1992) |
| Sce | 2C9, 2C19 | no | hamster/human, recon. | vitro | Goldstein et al. (1994) |
| Eco | 2D6 | yes | rabbit, rec. | vitro | Gillam et al. (1995) |
| Sce | several⁴ | no | yeast, coexp. | vitro | Imaoka et al. (1996) |
| Eco | 3A4 | yes | human, coexp. | vivo/vitro | Blake et al. (1996) |
| Eco | several⁵ | yes | human, coexp. | vivo/vitro | Iwata et al. (1998) |
| Sce | several⁶ | no | yeast/human, coexp. | vitro | Gervot et al. (1999) |
| Sce | several⁷ | no | human, coexp. | vitro | Masimirembwa et al. (1999) |
| Spo | 2C9 | no | human, coexp. | vitro | Takanashi et al. (2000) |
| Eco | 2B6 | yes | rat, rec. | vitro | Hanna et al. (2000) |
| Sty | several⁵ | no | human, coexp. | vitro | Fujita et al. (2001) |
| Ppa | 2D6 | no | human, coexp. | vitro | Dietrich et al. (2005) |
| Eco | 2B6 | yes | human, rec. | vitro | Mitsuda and Iwasaki (2006) |

¹“CYP” prefix skipped
²modification of the P450 amino acid sequence
³coexp. = coexpressed, rec. = reconstituted
⁴1A2, 2A6, 2B6, 2C8, 2C9, 2C19, 2D6, 2E1, 3A4
⁵1A1, 1A2, 1B1, 2C8, 2C9, 2C19, 2D6, 3A4, 3A5
⁶1A1, 1A2, 2B6, 2C8, 2C9, 2C19, 2D6, 3A4, 3A5
⁷1A1, 1A2, 2A6, 2C8, 2C9, 2C19, 2D6, 2E1, 3A4

### OBJECT OF THE INVENTION

Therefore, it is the object of the invention to establish an alternative recombinant expression system for the production of human liver P450s suitable for up-scaled biosynthesis of metabolites. Furthermore, it is an object of the invention to provide the method for the biosynthesis of pharmaceutically interesting metabolites depending on the cytochrome P450 metabolism.

### SUMMARY OF THE INVENTION

The invention therefore provides—iter alia—a recombinant fission yeast for the expression of cytochrome P450s comprising at least one expression cassette for the expression of a cytochrome P450 reductase (CPR) and, optionally, comprising one or more expression cassettes for the expression of cytochrome P450 (CYP). This expression can be due to a stable integration of the chosen sequences or due to the integration of said sequence into an extra-chromosomal DNA sequence, such as a plasmid.

The invention further provides recombinant fission yeast as above wherein the cytochrome P450 reductase (CPR) and the cytochrome P450 (CYP) are of mammalian or human origin. Preferably the sequence is selected from the sequences derived from pharmaceutically interesting mammals, such as e.g. pig, mouse, rat, ape, dog. The sequence is alternatively selected from human origin.

The invention further provides recombinant fission yeast as above wherein the human cytochrome P450 is selected from the group consisting of CYP2B6, CYP2D6, CYP2C9, CYP2C19 and CYP3A4.

The invention further provides recombinant fission yeast as above providing a production rate of CPR up to about 50 μmol/litre per day, preferably 75 μmol/litre per day, further preferably 100 μmol/litre per day, further preferably 200 μmol/litre per day.

The invention further provides recombinant fission yeast strain CAD62 comprising the sequence SEQ ID No: 1.

The invention further provides the use of recombinant fission yeast as above for the production of cytochrome P450, preferably human CPY450.

The invention further provides the use of recombinant fission yeast as above for the catalysation of cytochrome P450 dependent metabolic reactions and/or whole-cell biotransformation.

The invention further provides the use of recombinant fission yeast as above for the synthesis of at least one of the metabolites of the group consisting of 4-hydroxybupropion, hydroxymethyltolbutamid, 4-hydroxy-S-mephenytoin, dextorphan and 1′-hydroxymidazolam.

The invention further provides the method of producing at least one of the metabolites selected from the group consisting of 4-hydroxybupropion, hydroxymethyltolbutamid, 4-hydroxy-S-mephenytoin, dextorphan and 1′-hydroxymidazolam comprising the steps:
culturing of the recombinant fission yeast as above under suitable conditions;
incubating of the recombinant fission yeast with at least one substrate selected from the group consisting of bupropion, tolbutamide, S-mephenytoin, dextromethorphan and midazolam;
separating of the cell mass and the supernatant; and
isolating of the metabolite.

The invention further provides the method as above characterised in that the recombinant fission yeast as above provides a production rate of CPR up to about 50 μmol/litre per day, preferably 75 μmol/litre per day, further preferably 100 μmol/litre per day, further preferably 200 μmol/litre per day.

### DESCRIPTION OF THE INVENTION

The object of the invention is solved by the subject-matter of the independent claims. Additionally, the dependent claims refer to preferred embodiments.

### 2.1 RNA Sources

For the present invention cytochrome P450 (CYP 450) and cytochrome P450 reductase (CPR) derive from mammalian origin. According to one embodiment the CYP and/or CPR are selected from the known sequences for CYP and/or CPR deriving from ape, human, mouse, pig, dog, rat as well as other mammalians. According to a further embodiment the cDNA sequences of human CYP are selected from the group comprising the well described sequences CYP2B6, CYP2D6, CYP2C9, CYP2C19 and CYP3A4. According to a further embodiment the cDNA sequences of CPR are selected from the group comprising sequences retrieved from UniProtKB/Swiss-Prot (see Tab. 2), and than synthesised or optimized for fission yeast codon usage by Entelechon GmbH (Regensburg, Germany) or delivered as a pPCR-ScriptAmpSK(+) (Stratagene, La Jolla, Calif., USA) clones. All sequences were translated into amino acid code by using the free program BioEdit (author Tom Hall) and compared to the National Center for Biotechnology Information (NCBI) database using the BlastP server (Altschul et al., 1997). The synthesized cDNA was found to be 100% identical to reported sequences (see Tab. 2).

TABLE 2
Selected protein sequences used for the expression of human
liver P450s. Listed are synthesized human liver P450 nucleotide
sequences translated into amino acid sequences using
BioEdit and analyzed online using the NCBI Blast server.

| | EC | SwissProt | | Strain |
|---|---|---|---|---|
| Name | number | accession | Substrate | name |
| CYP2B6 | 1.14.14.1 | P20813 | bupropion | CAD65 |
| CYP2D6 | 1.14.14.1 | P10635 | tolbutamide | CAD68 |
| CYP2C9 | 1.14.13.80 | P11712 | mephenytoin | CAD66 |
| CYP2C19 | 1.14.13.80 | P33261 | dextromethorpan | CAD64 |
| CYP3A4 | 1.14.13.67 | P08684 | midazolam | CAD67 |
| CPR | 1.6.2.4 | P16435 | — | CAD62 |

### 2.2 Vectors

For expression in fission yeast the integrative vector pCAD1 (Dragan et al., 2005) and the autosomally replicating vector pREP1 (Maundrell, 1993) were chosen.

### 2.3 Expression Plasmid Construction

The pPCR-ScriptAmpSK(+) vectors bearing the desired cDNA sequences were subjected to a NdeI/BamHI double restriction and separated from the vector by gel electrophoresis. After DNA isolation from agarose gels, the cDNAs were ligated into in both, pCAD1 and pREP1. Analytical restrictions were done to assure correct cloning. All P450 cDNAs could be well distinguished by either HindIII, BglII, EcoRI and EcoRV fragmentation patterns.

General DNA manipulation methods were performed using standard techniques (Sambrook, et al., 1989). Media and genetic methods for studying fission yeast have been described in detail (Alfa, et al., 1993; Moreno, et al., 1991). Briefly, strains were generally cultivated at 30° C. in Edinburgh Minimal Media (EMM) with supplements of 0.1 g/l final concentration as required. Liquid cultures were kept shaking at 170 rpm. Thiamine was used at a concentration of 5 μM throughout.

### 2.4 Fission Yeast Strain Construction

Transformation of fission yeast was done either using cryocompetent cells (Suga and Hatakeyama, 2005) or the lithium acetate method (Okazaki et al., 1990). The strain NCYC2036 (MB163) with genotype h-ura4.dl18 and strain MB271 with genotype h-leu1.32. As can be seen in FIG. 3, fission yeast strains were constructed that either had a single copy of P450 cDNA chromosomally integrated and CPR added on a autosomally replicating vector or the other way round.

Additionally, strains were constructed that bear the respective P450 cDNA on an autosomally replicating vector. Testing of correct chromosomal integration of the pCAD1 plasmids was performed by plating colonies on EMM medium Petri dishes containing phloxine B.

Furthermore, strain MB163 (h-ura4.dl18) was transformed with pCAD1-CPR to yield strain CAD62. Correct chromosomal integration of the pCAD1-CPR construct into the leu1 locus was confirmed by plating colonies on medium lacking leucine EMM (Forsburg and Rhind, 2006). The resultant strain CAD62 was subsequently subjected to the cryo-preservation method (Suga and Hatakeyama, 2005). Cryo-preserved CAD62 cells were then transformed with pREP1 expression vectors bearing the respective P450 cDNAs to yield five different strains that coexpress CPR and one of the P450s (see Tab. 2).

### 2.5 Biomass Production

Cells of respective strains from permanent cultures were streaked on EMM dishes containing 0.01% leucine (w/v) and 5 μM thiamine and grown for 3 days at 30° C. Cell material from a plate was transferred to a 10 ml EMM preculture with 0.01% leucine (w/v) in absence of thiamine to induce the nmt1 promoter. From now on, all subsequent cultures were done in absence of thiamine to keep the promoter in an active state. Biomass production was scaled up by factor 10 to yield 100 ml or 1 L cell suspensions in batch cultures. Usually, the final cell density was around 5×10⁷ cells m⁻¹ with cells being in the stationary growth phase. After biomass production, the cells were centrifuged at 3000 g for 5 min except for 1 L cultures where centrifugation was carried out at 5000 g for 25 min. The biomass was washed three times with cold, deionized water and finally resuspended in certain volumes of 100 mM NaH₂PO₄/Na₂HPO₄ at indicated pH as required for the respective experimental conditions.

### 2.6 Activity Assays

Functional expression of all P450s was shown by incubation of constructed strains with recommended substrates (Walsky and Obach, 2004) as shown in Tab. 3. Substrate concentrations were always 1 mM, the incubation mixture consisted of 10 ml 10⁸ cells ml⁻¹ cell suspension suspended in sodium phosphate (NaPᵢ) buffer with pH=8.0.

Alternatively, following biomass production, the cells were centrifuged at 3000 g for 5 min. The biomass was washed with cold, deionized water and finally resuspended in the desired medium either being 100 mM sodium acetate/acetic acid medium for pH 3 to 5 or in 100 mM NaH₂PO₄/Na₂HPO₄ for pH 6, 7, 8, 9 and 10. Supplements like glucose and substrates were given as either aqueous or ethanolic solutions. The assay cell density was set to 2×10⁸ cells mL⁻¹ throughout. All assays were conducted in deep well microtiter well plates (dwMTP) with a nominal volume of 2 mL per well containing 1.1 mL of cell suspension and dissolved substrate during the assay. The used substrates were chosen according to Walsky and Obach (2004) and are listed in Tab. 3. The incubation time was 8 hours and the agitation was performed at 750 rpm using a shaker equipped with a MTP adapter.

TABLE 3
Used and generated fission yeast strains

| | | | | expression |
|---|---|---|---|---|
| strain | parent | protein | replication | cassettes per cell |
| CAD50 | MB163 | CYP2B6 | chromosomal | one |
| CAD57 | | CYP2C9 | | |
| CAD51 | | CYP2C19 | | |
| CAD49 | | CYP2D6 | | |
| CAD52 | | CYP3A4 | | |
| CAD62 | | CPR | | |
| CAD54 | CAD50 | CPR | autosomal | many |
| CAD55 | CAD51 | | | |
| CAD49 | CAD53 | | | |
| CAD52 | CAD56 | | | |
| CAD69 | MB271 | CYP2B6 | autosomal | many |
| CAD63 | | CYP2C9 | | |
| CAD70 | | CYP2C19 | | |
| CAD61 | | CYP2D6 | | |
| CAD59 | | CYP3A4 | | |
| CAD65 | CAD62 | CYP2B6 | autosomal | many |
| CAD68 | | CYP2C9 | | |
| CAD66 | | CYP2C19 | | |
| CAD64 | | CYP2D6 | | |
| CAD67 | | CYP3A4 | | |

### 2.7 Extraction of Metabolites

### 2.7.1 Bupropion

A volume 500 μl sample was applied on a HCX solid phase extraction cartridge and washed. A volume of 20 ml methanol-ammonia mixture (96:4 v/v) was three times applied in order to elute the metabolites, which were subsequently evaporated to dryness. The eluate was then reconstituted in the liquid phase and analyzed by LC-MS (liquid chromatography-mass spectroscopy) as described below (2.8.2).

### 2.7.2 Dextromethorphan and Midazolam

After incubation, the cells were separated from the supernatants by centrifugation at 5000 g for 20 min and 500 μl of supernatant were transferred to a 1.5 ml polypropylene reaction caps. After adding 250 pl of a mixture of 37% hydrochloric acid-2.3 M ammonium sulfate-10 M sodium hydroxide (1:2.5:1.5 v/v/v) and 500 μl of extraction solvent (dichloromethane-isopropanol-ethyl acetate, 1:1:3 v/v/v), the reaction caps were sealed and left on a rotary shaker for 1 min. After phase separation by centrifugation (10⁴ g, 2 min), the organic phase (upper) was transferred to an autosampler vial and evaporated to dryness under a stream of nitrogen at 56° C. The dry residues were acetylated with 100 μl of acetic anhydride-pyridine (3:2, v/v) for 5 min under microwave irradiation at 440 W. The excess reagent was evaporated and the residues were reconstituted in 100 μl of methanol. Aliquots (2 μl) of the acetylated extracts were analyzed by gas chromatography-mass spectrometry (GC-MS) as described.

### 2.7.3 Tolbutamide and Mephenytoin

After incubation, the cells were separated from the supernatants by centrifugation at 5000 g for 20 min and 500 μl of supernatant were transferred to a 1.5 ml polypropylene reaction caps. After adding a drop of 85% phosphoric acid and 500 μl of extraction solvent (ethylacetate-diethylether, 1:1 v/v), the reaction caps were sealed and left on a rotary shaker for 1 min. After phase separation by centrifugation (10⁴ g, 2 min), 400 μl of organic phase was transferred to an autosampler vial and evaporated to dryness under a stream of nitrogen at 56° C. Tolbutamide was methylated with 50 μl methanol and 100 μl diazomethane for 30 min under microwave irradiation at 440 W. For mephenytoin acetylation was carried as described above (2.7.2). The excess reagent was evaporated and the residues were reconstituted in 100 μl of methanol. Aliquots (2 μl) of the acetylated extracts were analyzed by gas chromatography-mass spectrometry (GC-MS) as described below.

### 2.8 Long-Term Incubations

To monitor the influence of temperature on the bioconversion rate assays were conducted in 250 mL erlenmeyer flasks filled with a cell suspension volume of 10 mL as described in principle (Dragan et al, 2005). The only difference consists in using sodium acetate/acetic acid or sodium phosphate buffered media as mentioned and in agitating at 150 rpm. Glucose and substrates were given as supplements. Initial activity tests were also done under these conditions whereby the pH was set to 8 for all strains.

### 2.9 Sample Preparation

All samples were centrifuged at 8000 g for 5 min to separate the biomass from the incubation supernatant. Before LC-MS/MS analyses, supernatants were transferred to autosampler vials and diluted 1:5 v/v with a mixture (60:40, v/v) of 50 mM aqueous ammonium formate adjusted to pH 3.5 with formic acid (eluent A):acetonitrile with 0.1% formic acid (eluent B). Aliquots (25 μL) of the diluted supernatants were injected into the LC-MS/MS system.

### 2.10 Analytics

### 2.10.1 Apparatus

The LC-MS/MS system was as follows: Shimadzu integrated HPLC system consisting of a Shimadzu CBM 20 A controller, two Shimadzu LC 20 AD pumps including a degasser, a Shimadzu SIL 20 AC autosampler, and a Shimadzu CTO 20 AC column oven; Applied Biosystems 3200 Q TRAP Linear Ion Trap Quadrupole Mass Spectrometer equipped with a Turbo V Ion Source operated in the electron spray ionization (ESI) mode and controlled by Analyst Software (Version 1.4.2).

2.10.2 LC conditions

The autosampler was cooled at 15° C. and operated without rinsing. Isocratic elution was performed on a GRACE mixed-mode C18/Cation 5u column (150 mm×4.6 mm internal diameter) as stationary phase. The column temperature was 30° C. The mobile phase composition (A:B, v/v) was 45:55 for analysis of dextromethorphan/dextrorphan and tolbutamide/HO-tolbutamide, 70:30 for analysis of bupropion/HO-bupropion, 35:65 mephenytoin/HO-mephenytoin, and 40:60 for analysis of midazolam and 1′-hydroxymidazolam. Flow rate and run time were 1 mL min-1 and 5 min, respectively. Before use, the mobile phases were degassed for 30 min in an ultrasonic bath. During use, the mobile phase was degassed by the integrated degasser. Before starting the analysis, the HPLC system was equilibrated for 10 min with the respective mobile phase composition.

### 2.10.3 Tandem MS Conditions

For detection and quantification, the following ESI inlet conditions were used: gas 1, nitrogen (50 psi; 344.7 kPa); gas 2, nitrogen (50 psi; 344.7 kPa); ion spray voltage, 5500 V; ion source temperature, 700° C.; curtain gas, nitrogen (10 psi; 68.9 kPa). The samples were analyzed in positive ion mode. The MS was operated in multiple-reaction monitoring (MRM) mode with Q1 and Q3 were operated in unit resolution with the following settings: collision gas was set at medium, the dwell time was set at 150 ms. All other settings were analyte-specific and were determined using Analyst software in quantitative optimization mode. Two transitions were monitored per analyte, one target transition (t) for quantification and one qualifier transition. The monitored transitions are reported in Table 3B.

### 2.10.4 GC-MS

The methylated or acetylated sample extracts were analyzed using a Hewlett Packard 296 (HP, Agilent Technologies, Waldbronn, Germany) HP 6890 Series GC system combined with an HP 5972 Series mass selective detector, an HP 6890 Series injector and an HP ChemStation G1701AA version A03.00. The GC conditions were as follows: splitless injection mode; column, HP-1 capillary (12 m×0.2 mm I.D.), cross linked methylsilicone, 330 nm film thickness; injection port temperature, 280° C.; carrier gas, helium; flow-rate 1 ml/min; column temperature, programmed from 100-310° C. at 30° C. min⁻¹, initial time 3 min, final time 8 min. The MS conditions were as follows: full scan mode, m z 50-550 u; EI ionization mode: ionization energy, 70 eV, ion source temperature, 220° C.; capillary direct interface heated at 260° C.

### 2.10.5 HPLC

A HP 1050 series HPLC system consisting of a quaternary pump, a degasser, an autosampler, variable wavelength (VWD) detector and an HP 1046A fluorescence detector was used. The stationary phase was a ZorbaxR 300-SCX column (2.1×150 mm, 5 μm). The mobile phase A consisted of 5 mmol/L ammonium formate buffer brought to pH 3 with formic acid. Mobile phase B consisted of acetonitrile containing 1% (v/v) formic acid. Samples were analyzed using an injection volume of 10 L of untreated incubation supernatant, a mobile phase composition of A:B 55:45 (v/v), a flow rate of 0.9 ml min⁻¹, ultraviolet (UV) detection at=265 nm, and a run time of 8 min. The mobile phases were and columns were: 70:30 v/v A:B for bupropion and 30:70 v/v A:B for tolbutamide on a Alltech mixed mode/cation exchange column.

### 3. Fission Yeast Strains Expressing Human Liver P450 and CPR

### 3.1 Strain Construction

Introducing the cDNA for the human CPR into the fission yeast genome was achieved by transforming the parent strain NCYC2036 with linearized pCAD1-hCPR. The resulting CAD62 strain clones were tested for correct integration by replica plating on EMM medium containing as supplements only thiamine and phloxine B. Correct integrands were chosen for

TABLE 3B
MRM transitions used in LC-ESI-MS/MS.

| | | m/z product ions |
|---|---|---|
| Analyte | m/z precursor ion | target ion (t) |
| dextrorphan | 258.23 | 157.1 |
| | | 199.2(t) |
| dextromethorphan | 272.262 | 171.3 |
| | | 213.5 |
| bupropion | 240.193 | 184.2 |
| | | 131.1 |
| hydroxybupropion | 256.182 | 238.6 |
| | | 139.1(t) |
| 4-hydroxymephenytoin | 235.168 | 150.2(t) |
| | | 105.1 |
| mephenytoin | 219.192 | 134.2 |
| | | 117.1 |
| midazolam | 326.118 | 292.1 |
| | | 249.7 |
| 1′-hydroxymidazolam | 342.131 | 325.2 |
| | | 203.2(t) |
| 4′-hydroxymidazolam | 342.121 | 326.2 |
| | | 298 |
| tolbutamide | 271.184 | 91.1 |
| | | 155.2 |
| 4-hydroxytolbutamide | 287.158 | 171.1(t) |
| | | 107.1 |

further use with one of them being subjected to the cryo-preservation procedure described by Suga and Hatakeyama (2005). After transforming cryo-preserved CAD62 with autosomally replicating pREP 1 plasmids bearing the optimized cDNAs for the mentioned human, microsomal P450 (see Tab. 2) clones were selected for an initial screen whereby a specific P450 in vivo activity could be detected. The strain growth was only mildly affected by over-expressing the microsomal P450 systems.

The strategy of strain construction further focused on the expression of each mentioned P450 isoenzymes (Tab. 3) alone. We chose two combinations regarding the number of heterologous expression cassettes per cell. One possibility was to express from the single-copy pCAD1 integrated into the genome (CAD49 to CAD52 and CAD57, Tab. 3) while the other was expression from the pREP1 multi-copy vector (CAD59, CAD61, CAD63, CAD69, CAD70, Tab. 3). The former strains grew well under standard conditions (EMM, 30 C, 150 rpm) and showed no altered phenotype when regarded through a phase-contrast microscope while the later appeared to grow significantly slower than wildtype.

However, later it became mandatory to coexpress the human CPR and the two strain sets were produced by either adding the CPR on a multi-copy plasmid to the integrated P450 (strains CAD53, CAD54, CAD55 and CAD56, CAD57 was not transformed anymore, Tab. 4) or by creating a new strain (CAD62) expressing CPR from a single-copy locus (using pCAD1). Strain CAD62 was subsequently used for the generation of a new set of strains expressing the coexpressed P450 from a multi-copy plasmid (CAD64 to CAD68). The phenotype of fully induced strains bearing the CPR expression cassette on a multi-copy plasmid showed severe growth problems and altered phenotypes (extremely dark coloured biomass, data not shown). These problems did not appear when the CPR was expressed from a single-copy cassette.

### 4. Functional Expression

### 4.1 P450 Activity

Testing the function of the heterologous liver P450s revealed a very low to missing activity in those strains that express P450s from a single-copy cassette with CAD49 showing the only significant bioconversion activity. Often, metabolite peaks in either GC-MS or HPLC were too low to be even qualitatively reliably detectable. We, therefore, decided to increase the expression level by constructing strains that express P450s from multi-copy plasmids. Only CAD61 showed a significant activity and CAD59 showed a very low activity. All other strains were not able to convert their substrate into the respective metabolite (see FIG. 1a-e). We decided to coexpress the human CPR in order to supply an electron donor to the host system that was able to actively cooperate with the expressed P450s. To do so, we transformed all fission yeast strains that expressed P450s from a single-copy expression cassette with the pREP1-CPR construct (multi-copy, autosomally replicating), however it soon became obvious that the overedxpression of human CPR from pREP1 inhibits growth. As described in 3.1, massive growth inhibitions resulted when the expression was induced via absence of thiamine and the cells could hardly reach 10⁷ cells ml⁻¹. This fact coupled with a missing activity towards the respective P450 substrates rendered this strategy not viable in light of a possible biotechnological use.

Therefore, we designed and constructed a new fission yeast strain that expresses the human CPR from the integrative single-copy plasmid pCAD1 (This strain was named CAD62). This strain was used as a platform for a new set of strains where P450 were additionally coexpressed from the multi-copy plasmid pREP1.

The resulting strains represent a new set of host systems where a functional, human electron transfer chain towards P450s could be established. This is supported by successfully demonstrating the bioconversion activity of all heterologously expressed P450s except for CAD68 (CYP2C9) where no hydroxylated product could be found (FIG. 2). However, as was found out later, this was due to an analytical problem, so that in the beginning the presence but also absence of activity could not be concluded. As in a parallel approach the analytical system was improved, and could be reliably determined for CYP2C9.

Furthermore, in a direct activity comparison with selected P450s and their standard substrates we could demonstrate a significant activity increase of up to about a factor of 3, or a factor of about 8 or even a factor of about 35 (see Tab. 4). This e.g. 3-fold, 8-fold and up to 35-fold increase of activity corresponds to a production rate of CPR between about 10-12 μmol/1 per day, about 18-22 μmol/1 per day, about 70-80 μmol/1 per day (Tab. 4).

TABLE 4
Fold increase in activity of human liver P450s heterologously expressed in fission yeast in
presence of coexpressed human CPR compared to strains expressing P450s alone. Activity assay
were carried out as described in 2.6. Supernatant samples at given time points were processed
and analyzed as indicated in 2.7 and 2.8. Rates were calculated by using the peak ratios of
product to substrate and converted to mole amount by multiplying with initial
concentrations (1 mM) and adjusted to 24 h (1 day = d).

| | | | rate at t = 47 h | [μmol L⁻¹d⁻¹] |
|---|---|---|---|---|
| Name | t = 19 h | t = 47 h | without CPR | with CPR |
| CYP2C19 | n.d.¹ | 35.7 | 0.35 | 12.5 |
| CYP2D6 | 2.6 | 3.0 | 23.8 | 71.7 |
| CYP3A4 | ∞² | 7.9 | 2.45 | 19.5 |

¹not determined
²due to missing activity in P450 alone strain

### 4.2 Influence of pH on Activity

Since we already knew that at least for a CYP2D6 expressing fission yeast strain the initial incubation pH of the buffer was affecting the production rate (Peters et al, 2007) we wanted to determine its influence on biotransformation by the new coexpressing strains. The measured metabolite concentrations in the supernatant of biomass incubations carried out in deep well MTPs were plotted against the initial buffer pH (FIG. 5). There are marked differences between low and high pH values with transitions of not detectable to significantly detectable metabolite concentrations occurring at 5<pH<6 for nearly all strains except for CAD65 expressing CYP2B6 (FIG. 5a) having its transition at 6<pH<7. The profile of biotransformation activity as a function of pH apparently differed dependent on the P450 isoform used. Two profile types can be recognized within the tested pH range. The first one can be characterized by a rise to a maximum activity level that afterwards remains constant (FIGS. 5a, c and e) while the other one peaks at a certain pH value and then decreases (FIG. 5d) or rises again at pH=10 (FIG. 5b). From these data we determined the apparent optimal pH values to be used in biotransformation as follows: pH=8 for bupropion conversion by CAD65, pH=7 for tolbutamide conversion by CAD68, pH=9 for mephenytoin conversion by CAD66, pH=7 for dextromethorphan conversion by CAD64 and pH=9 for the midazolam conversion by CAD76.

### 4.3 The Presence of Glucose

Having obtained the apparent optimal pH values the importance of glucose for the biotransformation was assessed. We set up a 2% glucose concentration in the buffer system which is the same as the one used in the EMM medium. The assay was carried out in deep well MTPs for 8 hours and the metabolite concentration in the supernatant was measured as described (FIG. 6). The results show that in principle the presence of glucose in the incubation medium does not significantly influence the rate of biotransformation except for strain CAD68 (expresses CYP2C9). While in absence of glucose only 1.4±0.3 μM of hydroxytolbutamide were found the presence of glucose significantly pushed the production to 3.4±0.6 μM (two-sided t-test, n=5, □=0.05, P=1.8×10₋₃).

### 4.4 Passive Gas Exchange

The P450 catalyzed reactions are dependent on oxygen which can be made available to the biotransforming cells by either assuring a passive gas exchange pathway between atmosphere and cell suspension or by actively pumping air through the culture by means of pumps in bioreactors. Oxygen is, however, also crucial for biomass growth and maintenance and a low concentration could possibly lead to competition between host and P450 demand. To test whether the presence of gas exchange during the growth and/or biotransformation period affects the biomass activity we cultured the strains in flasks where gas exchange was blocked during the whole growth period. The P450 activity of these cells was then compared to normally grown biomass (cellulose pot covered flasks). Furthermore, we investigated the influence of gas exchange on biotransformation in deep well MTPs by tightly sealing the wells and compared these activities with uncovered wells. For this assay we used normally grown biomass, as well. Blocked gas exchange during the biomass growth phase exerted a very diverse effect on the recombinant strains (FIG. 7). The most prominent negative impact was seen with strain CAD68 (expressing CYP2C19, two-sided t-test, n=5, □=0.05, P=5.4×10₋₄) showing an activity decrease of more than 300% while strain CAD66 expressing CYP2C19 showed a nearly 200% decrease (two-sided t-test, n=5, □=0.05, P=4.7×10₋₅). However, for strain CAD65 (CYP2B6 expressor) a slight but significant increase could be detected while for the CYP2D6 and CYP3A4 expressors only a moderate decrease was measured. The yield of metabolite in tightly sealed MTP wells was unexpectedly unaffected with strain CAD64 (CYP2D6 expressor) and only slightly diminished in CAD67 (CYP3A4 expressor). In return, for all other P450 expressors a significant decline in biotransformation activity was detected (two-sided t-test, n=5, □=0.05, P<10⁻⁶). Taken together, these data suggest a strain and possibly P450 isoform-dependent aeration effect that apparently did not affect the CYP2D6 and CYP3A4 expressors neither during biomass growth not throughout the bioconversion period (FIGS. 7d and e).

### 4.5 Temperature Effect in Long-Term Incubations

Since we expressed human enzymes in a cellular yeast environment we wanted to investigate the effect of rising the incubation temperature to 37° C. on activity. Thereby, in doing a longer monitoring we were able to record bioconversion over a period of 72 hours in most cases (FIG. 8). The picture here is as heterogeneous as in case of gas exchange. First of all, bioconversion at 30° C. could be detected in CAD65, CAD68 and CAD64 over the whole incubation period while in CAD65 a metabolite concentration of around 120 μM was reached. Strain CAD66 produced only metabolite at a concentration of less than 1 μM during the incubation period under decreasing activity. In case of strain CAD67 (CYP3A4, midazolam as substrate), however, bioconversion seems to halt after a couple of hours. A further observation made is that activities tend to be in general lower than in the MTP based assay (compare concentrations in FIG. 8 at t=7 h with FIG. 5, FIG. 6 and FIG. 7). Furthermore, at 30° C. most of the bioconversion activities were highest during the initial 24 hours while only for strain CAD68 a gradual activity increase was seen.

Second, the effect of elevated temperature on activity was most pronounced in CAD68 (CYP2C9, tolbutamide, FIG. 8b) where it led to a more than 200% increase in metabolite formation. Thereby, it can be clearly seen that the rate of metabolite formation after 24 hours is comparable to the one obtained at 30° C. after 48 hours. On the other hand, most affected in a negative way was the bioconversion activity of strain CAD66 showing a decreased metabolite formation by more than factor 2 when incubated at 37° C. (FIG. 8c). For the rest of strains notable positive changes in production were recorded during the initial 24 hours (see FIGS. 8a, d and e) while for the CYP3A4 expressor CAD67, although a higher concentration of metabolite appeared at 37° C., no information on kinetics can be extracted due to the very short period of activity.

As described above the use of recombinant in vivo system of the present invention might thus overcome the problems of the state of the art in that they are easier to handle while P450 expression and cofactor regeneration is done by the host. One major point in choosing the right organism is to ascertain the electron flow towards the terminal P450 by use of the required CPR. The invention provides a functional and purely human P450 system by coexpressing a CPR/P450 pair in fission yeast suitable for in vivo generation of P450 metabolites. In contrast to what was previously reported on biotransformation by a fission yeast strain solely expressing the human CYP2D6 (Peters et al, 2007), human CYP17 (Dragan et al, 2006b) and human CYP21 (Dragan et al, 2006a) the inventors saw the necessity to coexpress the human CPR since activity for CYP2B6, CYP2C9, CYP2C19 and CYP3A4 was barely detectable in strains without CPR coexpression.

This findings except for CYP2D6 are consistent with data reported on baker's yeast where CPR overexpression was crucial for the detection of P450 activity either in vivo (Truan et al, 1993) or in vitro (Peyronneau et al, 1992; Cheng et al, 2006). It is noteworthy that CPR expression was directed from an integrative element while P450 expression from an autosomal replicating plasmid. This lead to recombinant fission yeast strains that show a durable and reliable bioconversion activity even under harsh conditions like carrying out the bioconversion in sodium phosphate medium in absence of glucose, under high pH values or under elevated temperatures. The most pronounced effect on bioconversion was provoked by increasing the pH of the medium (FIG. 5), a fact that was already described by us (Peters et al, 2007). The bioinformatic analysis of the here used substrates by the online server based (http://www.vcclab.org/lab/alogps/start.html) ALOGPS 2.1 program (Tetko and Tanchuk, 2002) revealed pKa>5.0 for tolbutamide, pKa>6.0 for midazolam and pKa>7.5 for bupropion, (S)-mephenytoin and dextromethrophan. Based on the ability of fission yeast to sustain bioconversion under pH>6 and on basic thermodynamic theory we expected a higher pH to boost bioactivity by simply shifting the equilibrium of charged/uncharged substrate species towards the uncharged form, thereby, assuring a facilitated substrate passage through hydrophobic membrane cores. These expectations could be confirmed by our data (FIG. 5) although we are aware that they are not truly consistent with such a simple view. For instance the metabolite of dextromethorphan (pKa>8.0) shows a drop in concentration that started already at pH>7 and the metabolite of tolbutamide showed a similar drop at higher pHs (see FIGS. 5b and d). We also are aware that under the here used assay conditions the pH might be modified by the action of the fission yeast H+-ATPase encoded by pma1 (Ulaszewski et al, 1987) and the Na+/H+ antiporter encoded by sod2 (Jia et al, 1992). Since acidification of the medium is coupled to glucose availability (Ulaszewski et al, 1987) we expect the pH to remain more or less constant during an 8 hour period in absence of glucose. Moreover, Sod2 can work in reverse and by doing so, we frequently see a moderate pH increase after glucose is completely consumed and the buffer capacity exceeded (data not shown). However, the presence of glucose did definitely not alter the bioconversion activity of the strains except for CAD68 (CYP2C9) where a significant activity increase was recorded (FIG. 6). Taken together, our data demonstrate that the pH indeed is the major parameter that adjusts the bioconversion activity.

Next, the inventors inspected the influence of oxygen availability by blocking atmospheric gas exchange pathways during either culturing or bioconversion phases. Since the P450 reaction itself is dependent on oxygen, one would expect a drop in activity in its absence but this occurred only with three strains (FIGS. 7a, b and c). Noticeable, CAD64 (CYP2D6) and CAD67 (CYP3A4) were also unaffected when anaerobically grown (FIGS. 7d and e). The inventors have shown that expressing the human CYP21 in fission yeast led to an unexpectedly lowered oxygen consumption of the biomass in presence of substrate and assumed a possible competition situation between the two endogenous, microsomal P450s, CYP51 and CYP61, and the heterologously expressed P450 (Dragan et al, 2006a). It is inevitable that fission yeast produces ethanol when grown in presence of 20 g L₋₁ glucose and sealing gas exchange pathways leads to an even higher ethanol concentration. As a consequence, sterol biosynthesis is increased (Koukkou et al, 1993) which could interfere with electron availability for the expressed P450s. Since human CPR overexpression dramatically decreases fission yeast growth in a low P450 background (data not shown) we guess that the human reductase well interacts with the hosts P450s, thereby, possibly leading to a reduced flow of electrons towards the human P450s. The electron flow figure turns out to be a rather complex one including recent result from (Hughes et al, 2007) where the fission yeast hemoprotein Dap1 was shown to be positively implicated in microsomal P450 function in absence of oxygen and its human homologue, PGRMC1, to bind CYP3A4, CYP7A1 and CYP21. All these factors finally are highly dependent on the interaction probability between the host and the heterologously expressed P450 systems. Elevated temperatures might lead to an increased diffusion rate of substrate and to an increased effusion rate of metabolite, which would be a global effect. However, increasing the incubation temperature of bioconverting biomass to 37° C. (FIG. 8) led to an increased metabolite production of strains CAD65 (CYP2B6), CAD64 (CYP2D6) and partly CAD67 (CYP3A4) only during the first 24 hours while was disadvantageous in case of CAD66 (CYP2C19) and advantageous in case of CAD68 (CYP2C9). The diverse effect of elevated temperatures on P450 activity rather points towards a specific effect on the P450 isoform itself, which might outweigh the mentioned physical effect. This experiment, moreover, demonstrated that fission yeast can perform the desired P450 reactions for as long as 3 days in absence of glucose. Of course, measuring the metabolite concentrations in the biomass supernatant only gives a clue to what really happens during a bioconversion situation, which, finally, is the result of many superimposed rates and effects. For instance, vmax values measured with recombinant P450s revealed that differences are within an order of magnitude (Walsky and Obach, 2004) while Km values display a much wider range. For example, these values alone could not even explain why bupropion is so much faster converted than midazolam (compare FIGS. 8a to e). On the other hand, according to ALOGPS 2.1 solubility results for water the concentration of midazolam is around 30 μM and at pH=8.5 around 150 μM (Andersin, 1991) while for bupropion ALOGPS 2.1 delivers around 290 μM which is expected to be higher at the here used pH=8. Additionally, according to Tolle-Sander et al (2003) midazolam seems to be a very good substrate for human P-glycoprotein ATPases (ABCB1 subfamily) of which 5 homologue species exist in fission yeast (Iwaki et al, 2006) while bupropion is only a weak substrate (Wang et al, 2008). In case of midazolam, this could possibly lead to a low transient concentration at the subcellular location of the expressed P450, thereby, limiting activity. It is surprising to notice that only few heterologous P450 expression systems were specially designed for in vivo bioconversion purposes while one can find several publications concerned with the enzymatic characterization of recombinant P450s. Nevertheless, we compared the bioconversion activity of our system with some of the known human liver-P450 expression systems today whenever the same substrates were used. Escherichia coli was applied a couple of times in whole-cells assays obtaining very high rates (Blake et al, 1996; Iwata et al, 1998) when calculated per liter culture, however, a closer inspection of the conditions reveals the use of a low amount of cells and total volume (scaled in milliliters) and very short times (scaled in minutes). Of course, these are scales that are definitely not suitable and representative for the milligram scale bioproduction of P450 metabolites, a fact so clearly demonstrated by Vail et al (2005). In the later article the bacterial system only reaches a production rate of about 30% of 200 μM testosterone in 2 days in one liter of dense cell suspension in 100 mM potassium phosphate buffer supplied with 10 g glucose for several times. This turns to yield only around 30 μM day₋₁ a much lower number than our fission yeast production rate in EMM (20 g L₋₁ glucose) of about 450 _{K}M day₋₁ (data not shown). One can observe a fast initial bioconversion rate during the first two incubation hours followed by an activity stall for several hours indicating that activity is tightly linked to glucose presence while a low dissolved oxygen content (under 1%) must be guaranteed in order to significantly increase the yield on P450s. Bakers's yeast expression systems were introduced in the 1990s (Truan et al, 1993) but due to not matching in vivo data no reasonable comparison could be made. Recently, Pichia pastoris was tried as heterologous expression system for the human CYP2D6 but unfortunately in vivo data were not shown (Dietrich et al, 2005). A more promising approach was made in Sf21 cells infected by two baculovirus variants bearing either the P450 or the CPR cDNAs. When regarding the CYP3A4 testosterone hydroxylation rate of 90 μM day⁻¹ it is still significantly lower then our fission yeast rate.

A microbiological strain meant to produce P450 metabolites cannot be compared with strains normally used for mass production (e.g. ethanol, organic acids, secretory enzymes), however, it should be easy to handle and rugged. Our fission yeast based expression system can produce P450 metabolites after a biomass growth period without any need of additionally supply neither antibiotics, nor hemine, nor special inducers. The cells are kept in a fully induced expression state from the start on. Moreover, our recent results demonstrate that in principle a biotransformation can be carried out in the EMM medium without shifting the cells to a new environment (data not shown). More work will follow soon on exemplified bioconversions carried out in bioreactors since a little bit of control is finally needed.

### SHORT DESCRIPTION OF THE FIGURES

FIG. 1 shows chromatograms of activity assays done with fission yeast strains expressing human liver P450s from a multi-copy plasmid. Metabolites were extracted and analyzed as described above. Image (a) shows an HPLC chromatogram gained as described above while all other images show GC-MS chromatograms.

FIG. 2 shows chromatograms of activity assays done with fission yeast strains expressing the human CPR from a single-copy plasmid and human liver P450s from a multi-copy plasmid. Metabolites were extracted and analyzed as described above. Image (a) shows an HPLC chromatogram gained as described while all other images show GC-MS chromatograms.

FIG. 3 specifies for several validated P450 assays the selected human cytochrome, the recommended substrate, the type of reaction and the expected metabolite.

FIG. 4 shows a CPR cDNA sequence according to the invention.

FIG. 5 shows the dependence of metabolite formation by recombinant fission yeast strains on the initial medium pH. Means of double values with respective standard deviations gained by measuring the supernatant metabolite concentration are shown. Assay conditions: cell density was 2×10₈ cells mL₋₁ buffers were 100 mM sodium acetate/acetic acid for pH<6 or 100 mM NaH₂PO₄/Na₂HPO₄ for pH □5, incubation time was 8 h, substrate concentration was 1 mM for all, substrates were bupropion (CAD65).

FIG. 6 shows the influence of 2% glucose on metabolite production by recombinant fission yeast strains. Shown are mean values of measured metabolite concentrations in the supernatant with respective standard deviations (n=5). Assay conditions: cell density was 2×10₈ cells mL₋₁ buffers were 100 mM NaH₂PO₄/Na₂HPO₄ with an initial pH=8 (CAD65), pH=7 (CAD68), pH=9 (CAD66), pH=7 (CAD64) and pH=9 (CAD67), incubation time was 8 h, substrate concentration was 1 mM for all.

FIG. 7 shows the influence of gas exchange with the atmosphere on biotransformation. The left, dark columns of each subfigure indicate the concentration measured with cells grown in cellulose pot covered erlenmeyer flasks. The middle columns show values gained with cells grown in tightly sealed erlenmeyer flasks while the right, light columns display the metabolite concentration found in supernatants of cells that were grown with cellulose pots but were tightly sealed during the bioconversion period. Assay conditions: cell density was 2×10₈ cells mL₋₁ buffers were 100 mM NaH₂PO₄/Na₂HPO₄ with an initial pH=8 (CAD65), pH=7 (CAD68), pH=9 (CAD66), pH=7 (CAD64) and pH=9 (CAD67), incubation time was 8 h, substrate concentration was 1 mM for all.

FIG. 8 shows the influence of elevated incubation temperature on biotransformation. Assay conditions: cell density was 2×10₈ cells mL₋₁ buffers were 100 mM NaH₂PO₄/Na₂HPO₄ with an initial pH=8 (CAD65), pH=7 (CAD68), pH=9 (CAD66), pH=7 (CAD64) and pH=9 (CAD67), substrate concentration was 1 mM for all.

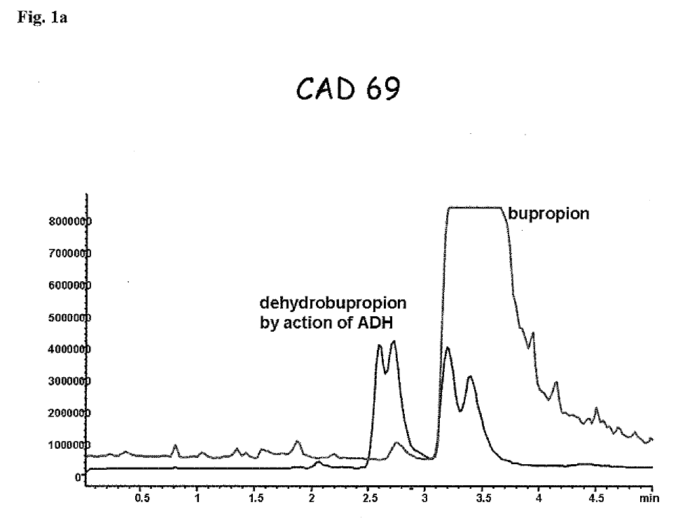

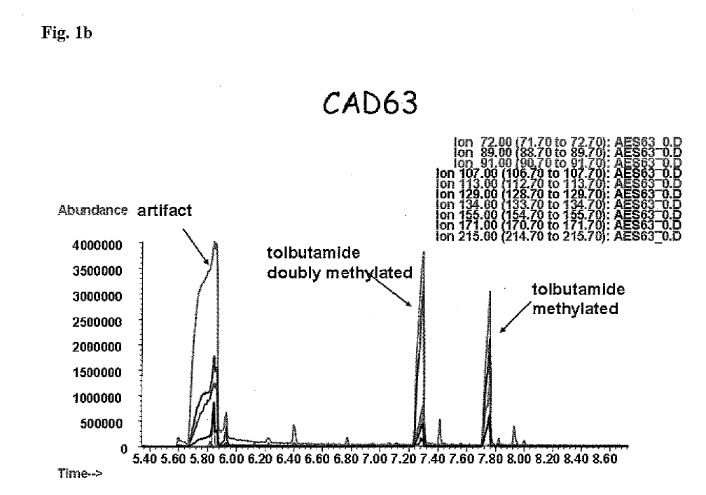

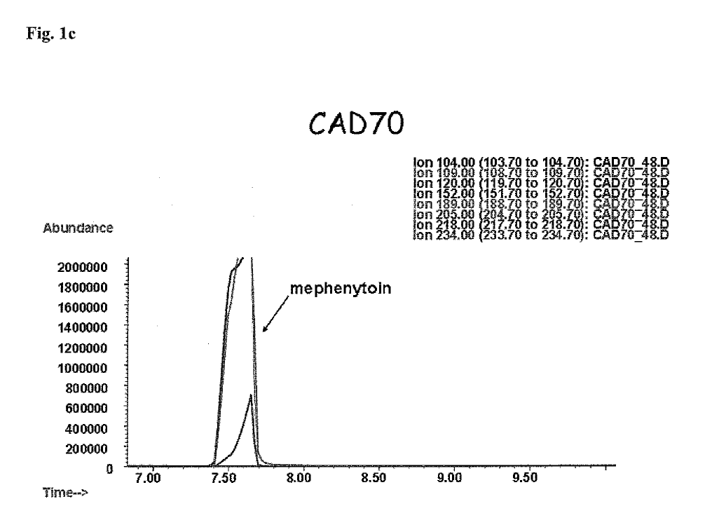

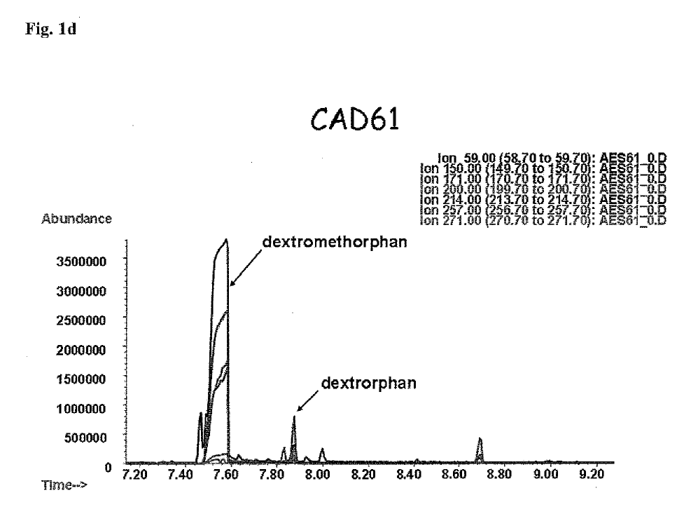

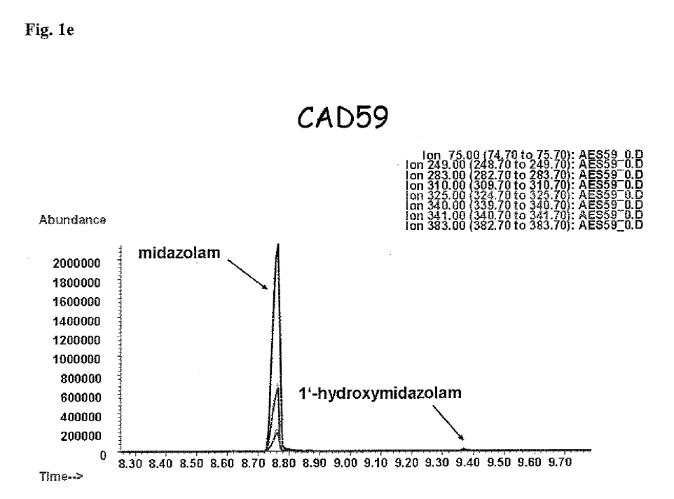

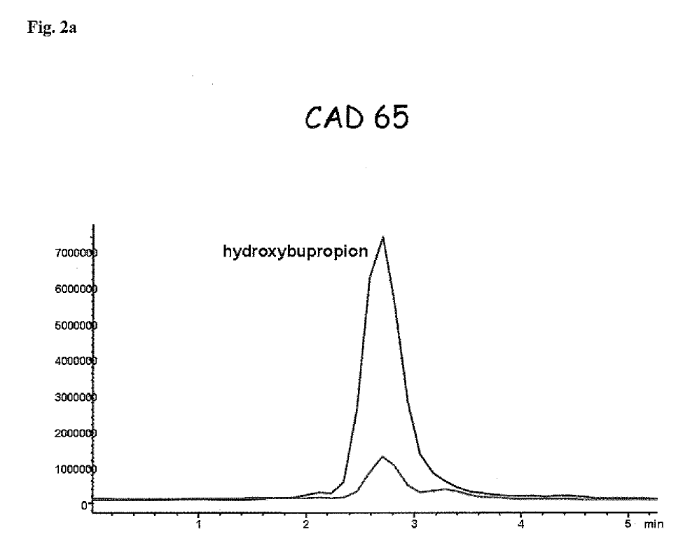

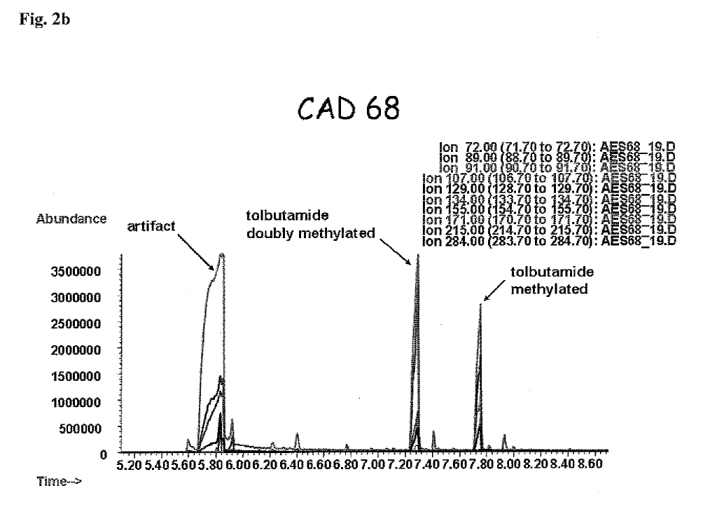

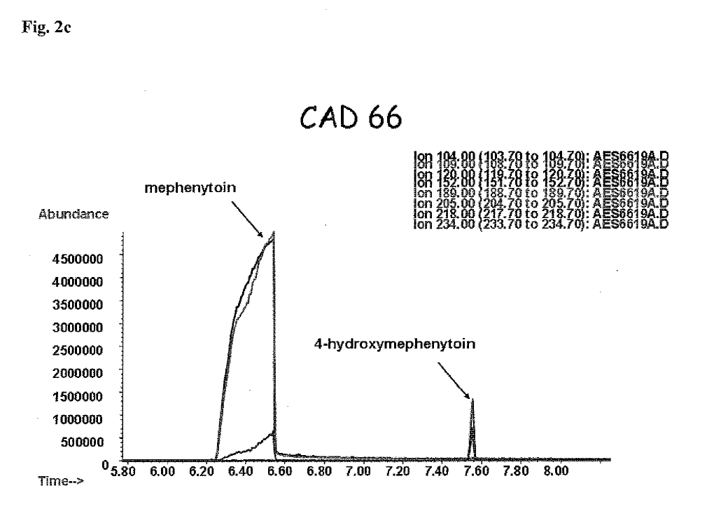

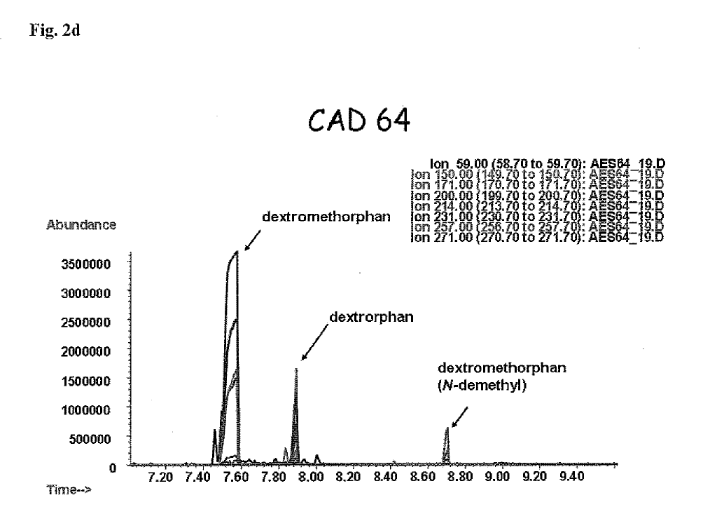

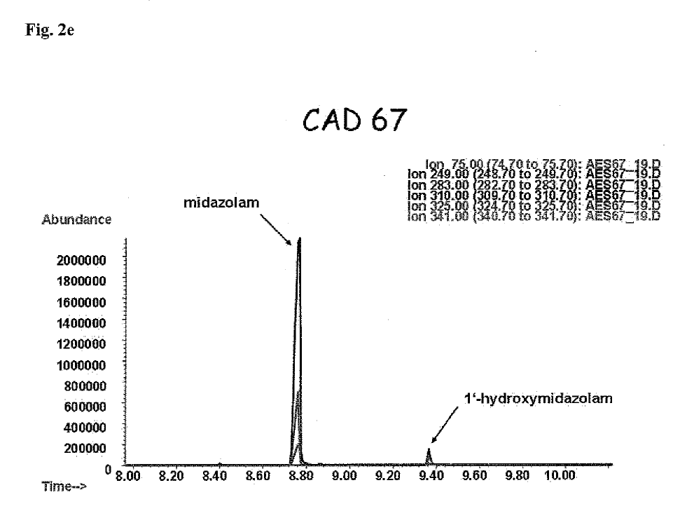

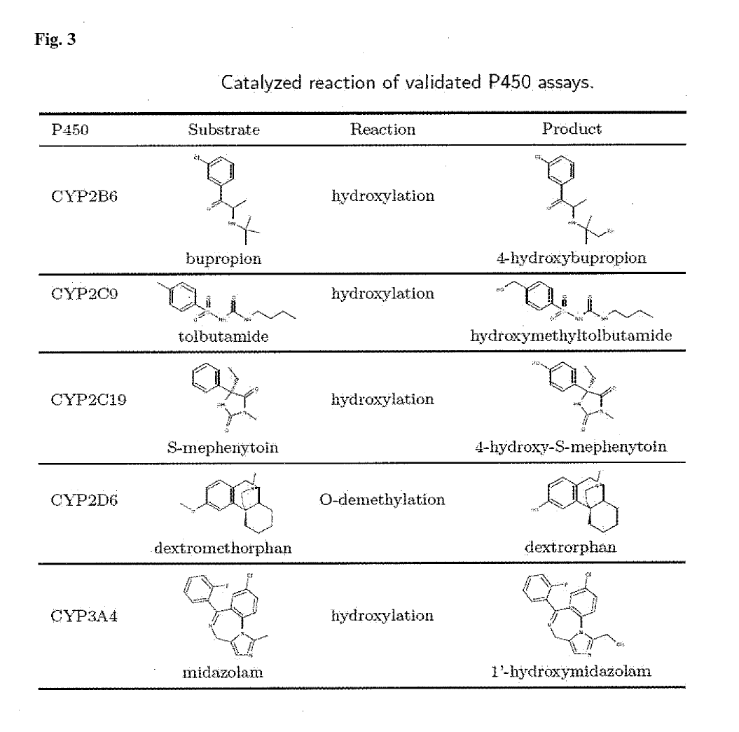

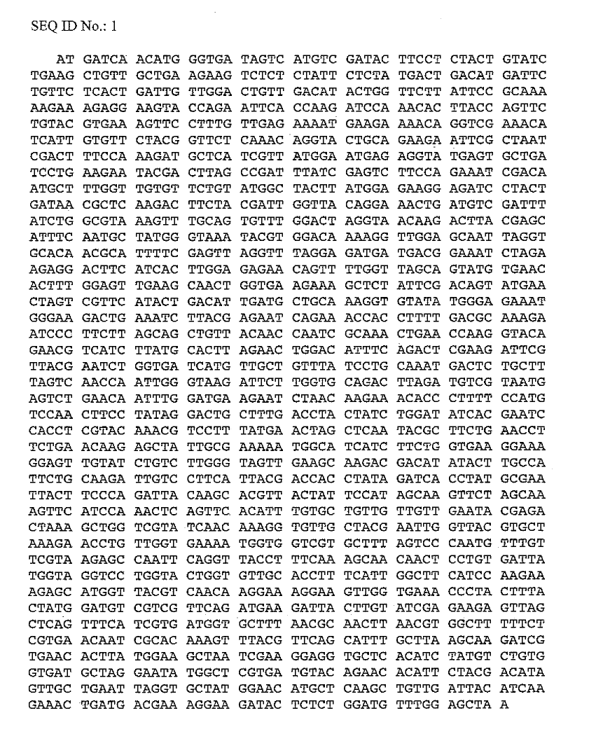

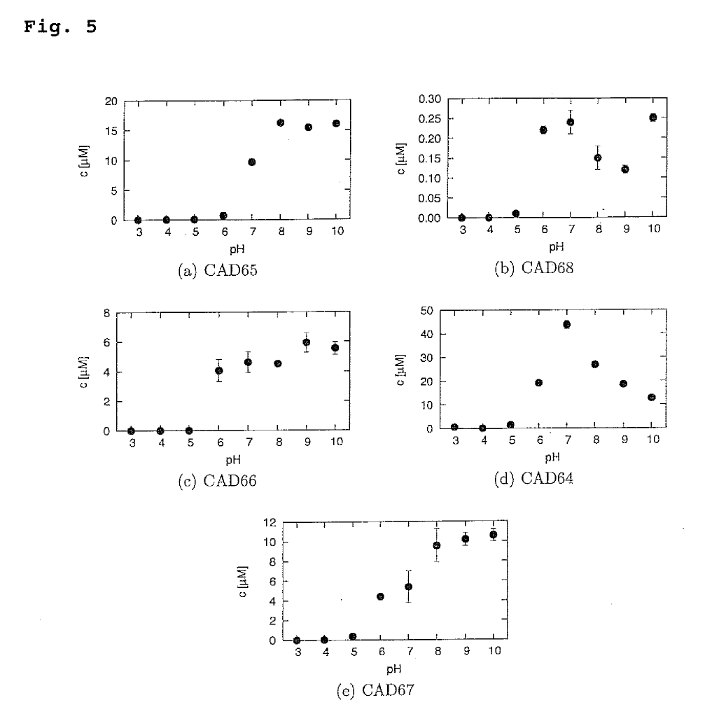

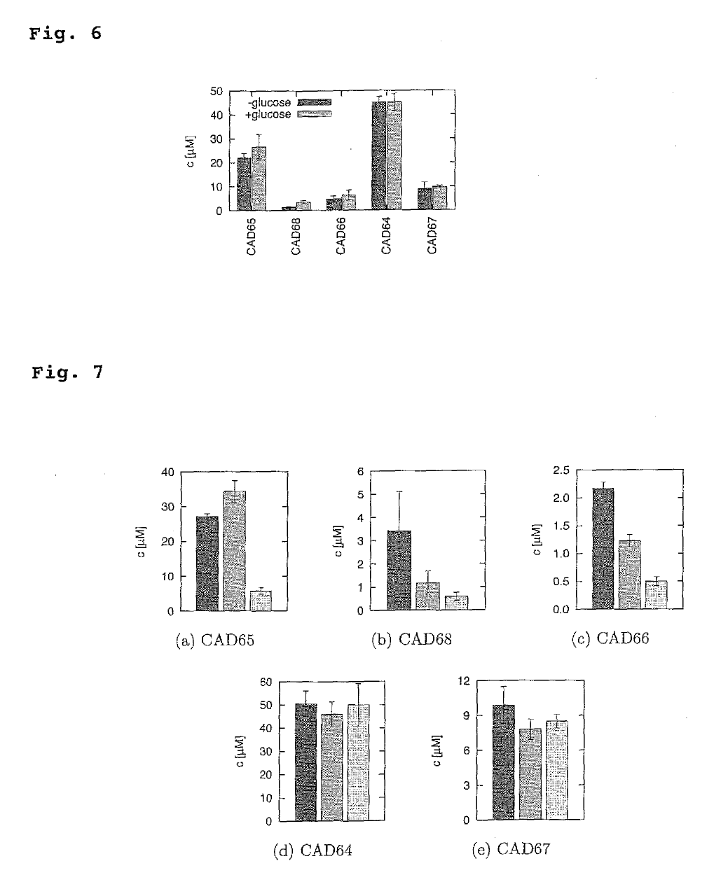

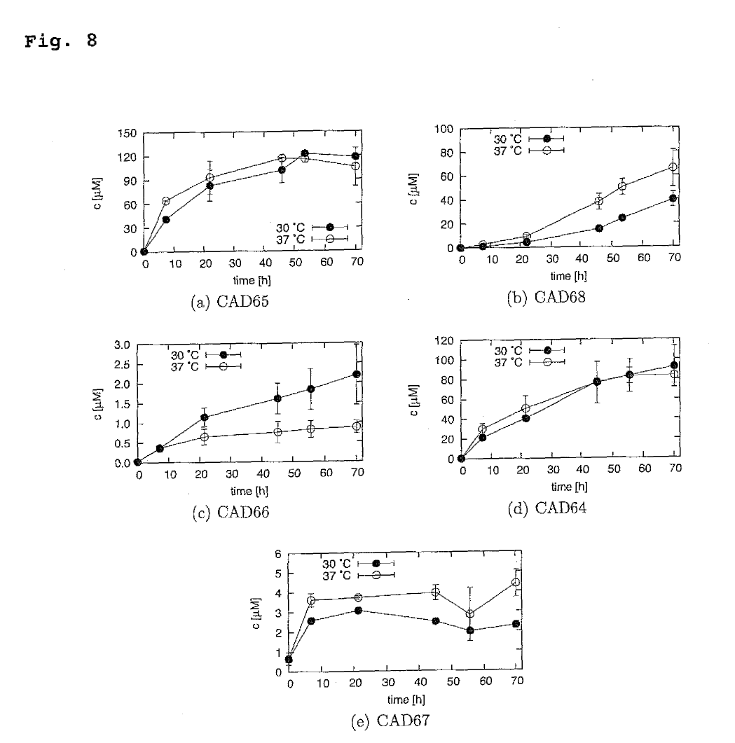

### REFERENCES

Altschul S F, Madden T L, Schaffer A A, Zhang J, Zhang Z, Miller W, Lipman D J (1997) Gapped blast and psi-blast: a new generation of protein database search programs. Nucleic Acids Res 25(17):3389-3402

Andersin R (1991) Solubility and acid-base behaviour of midazolam in media of different ph, studied by ultraviolet spectrophotometry with multicomponent software. J Pharm Biomed Anal 9(6):451-455

Blake J A, Pritchard M, Ding S, Smith G C, Burchell B, Wolf C R, Friedberg T (1996) Coexpression of a human p450 (cyp3a4) and p450 reductase generates a highly functional monooxygenase system in escherichia coli. FEBS Lett 397(2-3):210-214

Cheng J, fang Wan D, ren Gu J, Gong Y, li Yang S, cheng Hao D, Yang L (2006) Establishment of a yeast system that stably expresses human cytochrome p450 reductase: application for the study of drug metabolism of cytochrome p450s in vitro. Protein Expr Purif 47(2):467-476, DOI 10.1016/j.pep.2005.11.022, URL http://dx.doi.org/10.1016/j.pep.2005.11.022

Dietrich M, Grundmann L, Kurr K, Valinotto L, Saussele T, Schmid R D, Lange S (2005) Recombinant production of human microsomal cytochrome p450 2d6 in the methylotrophic yeast pichia pastoris. Chembiochem 6(11):2014-2022, DOI 10.1002/cbic.200500200, URL http://dx.doi.org/10.1002/cbic.200500200

Dragan C A, Zearo S, Hannemann F, Bernhardt R, Bureik M (2005) Efficient conversion of 11-deoxycortisol to cortisol (hydrocortisone) by recombinant fission yeast schizosaccharomyces pombe. FEMS Yeast Res 5(6-7):621-625, DOI 10.1016/j.femsyr.2004.12.001, URL http://dx.doi.org/10.1016/j.femsyr.2004.12.001

Dragan C A, Blank L M, Bureik M (2006a) Increased tca cycle activity and reduced oxygen consumption during cytochrome p450-dependent biotransformation in fission yeast. Yeast 23(11):779-794, DOI 10.1002/yea.1383, URL http://dx.doi.org/10.1002/yea.1383

Dragan C A, Hartmann R W, Bureik M (2006b) A fission yeast-based test system for the determination of ic50 values of anti-prostate tumor drugs acting on cyp21. J Enzyme Inhib Med Chem 21(5):547-556

Forsburg S L, Rhind N (2006) Basic methods for fission yeast. Yeast 23(3):173-183, DOI 10.1002/yea.1347, URL http://dx.doi.org/10.1002/yea.1347

Gillam E M, Guo Z, Martin M V, Jenkins C M, Guengerich F P (1995) Expression of cytochrome p450 2d6 in escherichia coli, purification, and spectral and catalytic characterization. Arch Biochem Biophys 319(2):540-550

Hanna I H, Reed J R, Guengerich F P, Hollenberg P F (2000) Expression of human cytochrome p450 2b6 in escherichia coli: characterization of catalytic activity and expression levels in human liver. Arch Biochem Biophys 376(1):206-216, DOI 10.1006/abbi.2000.1708, URL http://dx.doi.org/10.1006/abbi.2000.1708

Hughes A L, Powell D W, Bard M, Eckstein J, Barbuch R, Link A J, Espenshade P J (2007) Dap1/pgrmc1 binds and regulates cytochrome p450 enzymes. Cell Metab 5(2):143-149, DOI 10.1016/j.cmet.2006.12.009, URL http://dx.doi.org/10.1016/j.cmet.2006.12.009

Iwaki T, Giga-Hama Y, Takegawa K (2006) A survey of all 11 abc transporters in fission yeast: two novel abc transporters are required for red pigment accumulation in a schizosaccharomyces pombe adenine biosynthetic mutant. Microbiology 152(Pt 8):2309-2321, DOI 10.1099/mic.0.28952-0, URL http://dx.doi.org/10.1099/mic.0.28952-0

Iwata H, Fujita K, Kushida H, Suzuki A, Konno Y, Nakamura K, Fujino A, Kamataki T (1998) High catalytic activity of human cytochrome p450 co-expressed with human nadph-cytochrome p450 reductase in escherichia coli. Biochem Pharmacol 55(8):1315-1325

Jia Z P, McCullough N, Martel R, Hemmingsen S, Young P G (1992) Gene amplification at a locus encoding a putative na+/h+ antiporter confers sodium and lithium tolerance in fission yeast. EMBO J 11(4):1631-1640

Koukkou A I, Tsoukatos D, Drainas C (1993) Effect of ethanol on the sterols of the fission yeast schizosaccharomyces pombe. FEMS Microbiol Lett 111(2-3):171-175

Masimirembwa C M, Otter C, Berg M, Jönsson M, Leidvik B, Jonsson E, Johansson T, Bäckman A, Edlund A, Andersson TB (1999) Heterologous expression and kinetic characterization of human cytochromes p-450: validation of a pharmaceutical tool for drug metabolism research. Drug Metab Dispos 27(10):1117-1122

Maundrell K (1993) Thiamine-repressible expression vectors prep and prip for fission yeast. Gene 123(1):127-130

Mitsuda M, Iwasaki M (2006) Improvement in the expression of cyp2b6 by co-expression with molecular chaperones groes/el in escherichia coli. Protein Expr Purif 46(2):401-405, DOI 10.1016/j.pep.2005.10.017, URL http://dx.doi.org/10.1016/j.pep.2005.10.017

Okazaki K, Okazaki N, Kume K, Jinno S, Tanaka K, Okayama H (1990) High-frequency transformation method and library transducing vectors for cloning mammalian cdnas by trans-complementation of schizosaccharomyces pombe. Nucleic Acids Res 18(22):6485-6489

Peters F T, Dragan C A, Wilde D R, Meyer M R, Zapp J, Bureik M, Maurer H H (2007) Biotechnological synthesis of drug metabolites using human cytochrome p450 2d6 heterologously expressed in fission yeast exemplified for the designer drug metabolite 4′-hydroxymethyl-alpha-pyrrolidinobutyrophenone. Biochem Pharmacol 74(3):511-520, DOI 10.1016/j.bcp.2007.05.010, URL http://dx.doi.org/10.1016/j.bcp.2007.05.010

Peyronneau M A, Renaud J P, Truan G, Urban P, Pompon D, Mansuy D (1992) Optimization of yeast-expressed human liver cytochrome p450 3a4 catalytic activities by coexpressing nadph-cytochrome p450 reductase and cytochrome b5. Eur J Biochem 207(1):109-116

Suga M, Hatakeyama T (2005) A rapid and simple procedure for high-efficiency lithium acetate transformation of cryopreserved schizosaccharomyces pombe cells. Yeast 22(10):799-804, DOI 10.1002/yea.1247, URL http://dx.doi.org/10.1002/yea.1247

Tetko I V, Tanchuk V Y (2002) Application of associative neural networks for prediction of lipophilicity in alogps 2.1 program. J Chem Inf Comput Sci 42(5):1136-1145

Tolle-Sander S, Rautio J, Wring S, Polli J W, Polli J E (2003) Midazolam exhibits characteristics of a highly permeable p-glycoprotein substrate. Pharm Res 20(5):757-764

Truan G, Cullin C, Reisdorf P, Urban P, Pompon D (1993) Enhanced in vivo monooxygenase activities of mammalian p450s in engineered yeast cells producing high levels of nadph-p450 reductase and human cytochrome b5. Gene 125(1):49-55

Ulaszewski S, Herck J C V, Dufour J P, Kulpa J, Nieuwenhuis B, Goffeau A (1987) A single mutation confers vanadate resistance to the plasma membrane h+-atpase from the yeast schizosaccharomyces pombe. J Biol Chem 262(1):223-228

Vail R B, Homann M J, Hanna I, Zaks A (2005) Preparative synthesis of drug metabolites using human cytochrome p450s 3a4, 2c9 and 1a2 with nadph-p450 reductase expressed in escherichia coli. J Ind Microbiol Biotechnol 32(2):67-74, DOI 10.1007/s10295-004-0202-1, URL http://dx.doi.org/10.1007/s10295-004-0202-1

Walsky R L, Obach R S (2004) Validated assays for human cytochrome p450 activities. Drug Metab Dispos 32(6):647-660, DOI 10.1124/dmd.32.6.647, URL http://dx.doi.org/10.1124/dmd.32.6.647

Wang J S, Zhu H J, Gibson B B, Markowitz J S, Donovan J L, DeVane C L (2008) Sertraline and its metabolite desmethylsertraline, but not bupropion or its three major metabolites, have high affinity for p-glycoprotein. Biol Pharm Bull 31(2):231-234

1. A recombinant fission yeast for the expression of cytochrome P450s comprising at least one expression cassette for the expression of a cytochrome P450 reductase (CPR) and, optionally, comprising one or more expression cassettes for the expression of cytochrome P450 (CYP).

2. The recombinant fission yeast according to claim 1, wherein the cytochrome P450 reductase (CPR) and the cytochrome P450 (CYP) are of mammalian or human origin.

3. The recombinant fission yeast according to claim 1, wherein the human cytochrome P450 is selected from the group consisting of CYP2B6, CYP2D6, CYP2C9, CYP2C19 and CYP3A4.

4. The recombinant fission yeast according to claim 1, providing a production rate of CPR up to about 100 μmol/litre per day.

5. The recombinant fission yeast strain CAD62 comprising the sequence SEQ ID No: 1.

6. A method for the production of human cytochrome P450, said method comprising culturing the recombinant fission yeast according to any of claim 1 or 5 under suitable conditions so as to produce human cytochrome P450.

7. A method for the catalysation of cytochrome P450 dependent metabolic reactions and/or whole-cell biotransformation, said method comprising culturing the recombinant fission yeast according to any of claim 1 or 5 in the presence of a substrate and under suitable conditions for the catalysation of cytochrome P450 dependent metabolic reactions and/or whole-cell biotransformation of the substrate.

8. A method for the synthesis of at least one of the metabolite, said method comprising culturing the recombinant fission yeast according to any of claim 1 or 5 in the presence of a substrate and under suitable conditions, wherein said metabolite is selected from of the group consisting of 4-hydroxybupropion, hydroxymethyltolbutamid, 4-hydroxy-S-mephenytoin, dextorphan and 1′-hydroxymidazolam.

9. A method of producing at least one metabolite selected from the group consisting of 4-hydroxybupropion, hydroxymethyltolbutamid, 4-hydroxy-S-mephenytoin, dextorphan and 1′-hydroxymidazolam, said method comprising the steps of:
(a) culturing of the recombinant fission yeast according to any of the claim 1 or 5 under suitable conditions;
(b) incubating of the recombinant fission yeast with at least one substrate selected from the group consisting of bupropion, tolbutamide, S-mephenytoin, dextromethorphan and midazolam;
(c) separating of the cell mass and the supernatant; and
(d) isolating of the metabolite.

10. The method according to claim 8, wherein the recombinant fission yeast provides a production rate of CPR up to about 100 μmol/litre per day.
